(19) United States
(12) Patent Application Publication
(10) Pub. No.: US 2005/0002987 A1
(43) Pub. Date: Jan. 6, 2005

(54) Transnasal microemulsions containing diazepam

(76) Inventors: Yong Moon Choi, Pine Brook, NJ (US); Kwon H. Kim, Palisades Park, NJ (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/869,195

(22) Filed: Jun. 16, 2004

Related U.S. Application Data
(60) Provisional application No. 60/479,281, filed on Jun. 17, 2003.

(51) Int. Cl. A61K 9/70; A61K 9/14

(52) U.S. Cl. 424/434; 424/489

(57) ABSTRACT

Diazepam is administered intranasally in the form of specific microemulsions having advantageous properties. The microemulsions are comprised of about equal quantities of a fatty acid and water with the remainder being a hydrophilic surfactant, a polar solvent and an alcohol in a weight ratio such that alcohol is present in a greater quantity by weight than either of the other two. Nasal administration of the subject microemulsions produces a high plasma concentration of diazepam nearly as fast as intravenous administration. The present microemulsions are particularly suitable for a prompt and timely treatment of patients in the acute and/or emergency treatment of status epilepticus and other fever-induced seizures.

### RELATED APPLICATIONS

This application is a continuation of U.S. Provisional Application No. 60/479,281, filed Jun. 17, 2003.

### FIELD OF THE INVENTION

The present invention is directed to pharmaceutical compositions for transmucosal delivery of diazepam.

### BACKGROUND OF THE INVENTION

Status epilepticus is a neurological emergency in which mortality ranges from 3-35%. The major goal of treatment is rapid management of pathological seizure activity; the longer that the episode of status epilepticus is untreated, the more difficult it is to control and the greater the risk of permanent brain damage. Thus, critical to the management of the patient is a clear plan, involving prompt treatment with effective drugs in adequate doses having a proper pharmaceutical formulation as well as attention to hypoventilation and hypotension.

Currently several drug regimens have been proven to be applicable in treating status epilepticus. Diazepam is one of the most widely used benzodiazepines for this purpose. Intravenous administration of anticonvulsants is the most rapid way to suppress epileptic convulsions. However, other routes of administration may be highly desirable when intravenous administration is inconvenient and delaying, for instance, because of technical difficulties such as requirements for sterile equipment and skilled personnel, and because of the possible development of thrombophlebitis. In addition, intravenous medication is often associated with hypotension, cardiac dysrhythmia or central nervous system depression. In this regard Moolenaar et al., Int. J. Pharm., 5: 127-137 (1986), attempted to administer diazepam in humans via several other routes such as intramuscular injection, oral tablet and rectal solution. Only the rectal administration was found to provide a fairly rapid absorption and thus, it might be looked upon as an alternative route to IV injection. However, the rectal route is a very inconvenient way of drug administration particularly in emergency treatment. In Burghardt, U.S. Pat. No. 4,863,720, a sublingual sprayable pharmaceutical preparation is disclosed, in which the active drug can be a benzodiazepine, optimally comprising polyethylene glycol (PEG) and requiring ethanol, a di- and/or triglyceride of fatty acids and a pharmaceutically acceptable propellant gas.

More recently, it appears that the mucosal membrane of the nose offers a practical route of administration for therapeutic effect of many medicinal substances. Intranasal administration has the advantages that drugs may be administered readily and simply to achieve a systemic or localized effect, as required. However, the major problem associated with intranasal drug administration is the fact that most drug molecules diffuse poorly and slowly through the nasal mucosal membrane and thus the desired levels of the therapeutic agent cannot be achieved by means of simple transnasal administration. An additional constraint concerning nasal administration is that administration is limited to a small volume, i.e. it is generally not possible to administer more than approximately 150 μl per nostril. Volumes of formulation above this level will drain out into the pharynx and be swallowed.

Therefore, a great need exists for solvent vehicles which dissolve the desired medication, i.e. diazepam, to a high concentration, yet which are not irritating to the nasal mucosa. The intranasal absorption of drugs can be increased by coadministering a chemical adjuvant or permeation enhancers. For example, Lau and Slattery [Lau et al., Int. J. Pharm., 54: 171-174 (1989)] attempted to administer a benzodiazepine such as diazepam by dissolving it in a variety of solvents; triacetin, dimethylsulfoxide, PEG 400, Cremophor EL, Lipal-9-LA, isopropyl adipate and Azone. While many of the solvents dissolved diazepam in the desired concentrations, they were too irritating to be used for transnasal administration. Cremophor EL was found to be the least irritating for nasal mucosal tissue, but the nasal absorption in the use of this vehicle in humans was rather slow (Tₘₐₓ≅1.4 hours) and the peak concentration was low relative to that observed after IV administration.

A transnasal solution possessing enhanced properties is described in our co-pending patent application Ser. No. 09/624,305. The carrier for this solution is an aqueous vehicle containing an aliphatic alcohol having 1 to 5 carbon atoms, a glycol such as propylene glycol and a biological surfactant selected from bile salts and lecithins. These solutions preferably contain equal quantities of the alcohol and glycol. Such solutions have been shown to be effective for the transnasal administration of certain medicaments, particularly the benzodiapines. More recently, Li et al. International Journal of Pharmaceutics Vol. 237, pp 77-85, 2002, described microemulsions for rapid-onset transnasal delivery of diazepam. Microemulsions of diazepam similar to those described by Li et al. but having enhanced properties are provided in accordance with the present invention.

### SUMMARY OF THE INVENTION

In accordance with the present there are provided novel microemulsion formulations containing diazepam. Diazepam is administered intranasally in the form of specific microemulsions having advantageous properties over similar compositions disclosed in the literature. The microemulsions are comprised of about equal quantities of a fatty acid ester and water with the remainder being a hydrophilic surfactant, a polar solvent and an alcohol, preferably an aliphatic alcohol, in a weight ratio such that alcohol is present in a greater quantity by weight than either of the other two. Nasal administration of the subject microemulsions produces a high plasma concentration of diazepam nearly as fast as intravenous administration. The present microemulsions are particularly suitable for a prompt and timely treatment of patients in the acute and/or emergency treatment of status epilepticus and other fever-induced seizures.

### DETAILED DESCRIPTION OF THE INVENTION

In accordance with the present invention, there are provided microemulsion formulations containing diazepam that are advantageous in comparison to the microemulsion formulations described in the literature. The microemulsion diazepam formulations described by Li et al. are characterized by an ethyl laurate content of about about 15 wt. percent and a comparable content of water. The formulations are otherwise comprised of a aliphatic alcohol, i.e. ethanol, a glycol, i.e. propylene glycol and polysorbate 80, i.e. polyoxyethylene (20) sorbitan mono-oleate. In one of the formulations disclosed by Li et al., the concentration of ethanol is about one quarter of that of each of the glycol and polysorbate 80. In the other, the weight ratio of the three components is the same. It is stated therein that the formulation wherein the weight ratio of alcohol, glycol and polysorbate 80 is 1:1:1 is superior to that having a reduced alcohol content in comparison to than of the glycol and polysorbate 80.

In accordance with the present invention, there are provided formulations that have a decreased proportion of polar solvent, e.g. glycol, to even the enhanced formulation described by Li et al. Further in contrast to the formulations described by Li et al., the formulations of the present invention are characterized by an alcohol content that is greater than either of the polar solvent content and the hydrophilic surfactant (e.g. polysorbate 80) content. These formulations demonstrate enhanced properties with regard to at least one of several criteria, such as those described by Li et al., i.e. rapidity of onset of activity, solubilization of the diazepam, particle size analysis and in vivo absorption. Formulations of the present invention contain approximately equal quantities of water and a fatty acid ester, preferably not less than 10 percent each, more preferably about 10 to about 25 weight percent each and most preferably about 15 weight percent of each with the reminder being comprised of a hydrophilic surfactant, a polar solvent and an alcohol, wherein, in the weight ratio of the three, the alcohol is always greater than either of the other two. Suitable fatty acid esters include but are not limited to ethyl laurate, ethyl myristate, ethyl palmitate, ethyl linoleate, propyl isobutylate, isopropyl laurate, isopropyl myrisate, and combinations thereof. A particularly preferred fatty acid ester is ethyl laurate. Suitable hydrophilic surfactants include but are not limited to TWEEN 80(POLYSORBATE 80) TWEEN 20, 40, 60 and combinations thereof. Suitable polar solvents include but are not limited to propylene glycol, polyethylene glycols such as PEG 300, PEG 400, PEG 600 and combinations thereof. Particularly preferred alcohols include the lower alkanols such as ethanol or isopropanol. Essentially any aliphatic alcohol having from 2 to 12 and more preferably, from 2 to 8 carbon atoms can be employed. A particularly preferred example of a suitable alcohol is ethanol.

In one preferred embodiment, formulations of the present invention contain equal quantities of water and ethyl laurate, preferably about 15 weight percent of each with the reminder being comprised of polysorbate 80, propylene glycol and ethanol wherein, in the weight ratio of the three, the ethanol is always greater than either of the other two.

### EXAMPLES

Exemplary ethyl laurate-containing formulations of the present invention may comprise polysorbate 80, propylene glycol:ethanol weight ratios of 1.0:0.86:1.15; 1.0:0.72:1.29 and 1.0:1.0:1.5. Specific exemplary formulations are set forth in Table 1, wherein each component is given in percent weight to weight. These examples serve to illustrate but do not limit the invention described herein.

TABLE 1

| | Formula A | | |
|---|---|---|---|
| Component | (% w/w) | Formula B (% w/w) | Formula C (% w/w) |
| Ethyl Laurate | 15.0 | 15.0 | 15.0 |
| Polysorbate 80 | 23.3 | 23.3 | 20.0 |
| Propylene Glycol | 20.0 | 16.7 | 20.0 |
| Ethanol | 26.7 | 30.0 | 30.0 |
| Water | 15.0 | 15.0 | 15.0 |

The subject emulsions are formed by conventional techniques. The diazepam is initially dissolved in the ethyl laurate, which is the oil phase of the emulsions. The emulsions appear to be bicontinuous systems and are characterized by having good sprayability due in part to the increased ethanol content. Diazepam will dissolve in the subject emulsions to a concentration of about 40 mg./ml. Hence, it is possible to administer a therapeutic dosage of diazepam via intranasal administration by one to two sprays per nostril from a suitable conventional spray device which would constitute from about 250 to 500 microliters of microemulsion.

From a clinical point of view, intranasal administration often provides an improved duration of anticonvulsive effect. Therefore, the microemulsions of the present invention are advantageous for the treatment of status epilepticus and other conditions where the rapid suppression of convulsions is required. The increased water content of the subject emulsions in comparison to the solutions described above provides for a lesser incidence of nasal irritation. Although this invention has been described with respect to the therapeutic application of diazepam as an anticonvulsant, it is understood that the subject emulsions are also applicable to the other recognized therapeutic indications of diazepam.

The present invention is not to be limited in scope by the specific embodiments describe herein. Indeed, various modifications of the invention in addition to those described herein will become apparent to those skilled in the art from the foregoing description. Such modifications are intended to fall within the scope of the appended claims.

1. A microemulsion for the transnasal administration of diazepam comprising an emulsion vehicle containing water, a fatty acid ester, a hydrophilic surfactant, a polar solvent and an alcohol, wherein the fatty acid and the water are present in the vehicle in about equal amounts and wherein the alcohol is present in an amount greater than either one of the hydophilic surfactant and the polar solvent.

2. A microemulsion in accordance with claim 1, wherein the fatty acid ester is selected from the group consisting of ethyl laurate, ethyl myristate, ethyl palmitate, ethyl linoleate, propyl isobutylate, isopropyl laurate, isopropyl myrisate, and combinations thereof.

3. A microemulsion in accordance with claim 1, wherein the hydrophilic surfactant is selected from the group consisting of polysorbate 80, Tween 20, 40, 60 and combinations thereof.

4. A microemulsion in accordance with claim 1, wherein the polar solvent is selected from the group consisting of propylene glycol, polyethylene glycol and combinations thereof.

5. A microemulsion in accordance with claim 4, wherein the polyethylene glycol is selected from the group consisting PEG 300, PEG 400 PEG 600 and combinations thereof.

6. A microemulsion in accordance with claim 1, wherein the alcohol is selected from a group consisting of ethanol, isopropanol and combinations thereof.

7. A microemulsion in accordance with claim 1, wherein the fatty acid ester and the water each comprise not less than 10 percent by weight of the vehicle.

8. A microemulsion in accordance with claim 1, wherein the fatty acid ester and the water each comprise from about 10 to about 25 percent by weight of the vehicle.

9. A microemulsion in accordance with claim 1, wherein the fatty acid ester and the water each comprise about 15 percent by weight of the vehicle.

10. A microemulsion in accordance with claim 1, containing about 20 weight percent of said hyrophilic surfactant and the weight ratio of the hydophilic surfactant:polar solvent:alcohol is about 1.0:1.0:1.5.

11. A microemulsion in accordance with claim 1, wherein the emulsion vehicle contains ethyl laurate, polysorbate 80, propylene glycol, ethanol and water wherein each of ethyl laurate and water comprise 15 percent by weight of the vehicle, and the weight ratio of polysorbate 80, propylene glycol and ethanol is such that the proportion of alcohol is greater than either of the other two.

12. A microemulsion in accordance with claim 11, containing about 23.3 weight percent of polysorbate 80 and the weight ratio of polysorbate 80, propylene glycol and ethanol is 1.0:0.86:1.15.

13. A microemulsion in accordance with claim 11, containing about 23.3 weight percent of polysorbate 80 and the weight ratio of polysorbate 80, propylene glycol and ethanol is 1.0:0.72:1.29.

14. A microemulsion in accordance with claim 11, containing about 20 weight percent of polysorbate 80 and the weight ratio of polysorbate 80, propylene glycol and ethanol is 1.0:1.0:1.5.

15. In a method of administering diazepam to a patient in need thereof via transnasal administration, the improvement wherein diazepam is administered in a microemulsion vehicle in accordance with claim 1.

16. A method in accordance with claim 15, wherein the fatty acid ester and the water each comprise from about 10 to about 25 percent by weight of the vehicle.

17. A method in accordance with claim 15, wherein the fatty acid ester and the water each comprise from about 10 to about 25 percent by weight of the vehicle.

18. A method in accordance with claim 15, wherein the fatty acid ester is selected from the group consisting of ethyl laurate, ethyl myristate, ethyl palmitate, ethyl linoleate, propyl isobutylate, isopropyl laurate, isopropyl myrisate, and combinations thereof; the hydrophilic surfactant is selected from the group consisting of polysorbate 80, Tween 20, 40, 60 and combinations thereof; the polar solvent is selected from the group consisting of propylene glycol, polyethylene glycol and combinations thereof; the polyethylene glycol is selected from the group consisting PEG 300, PEG 400 PEG 600 and combinations thereof; and the alcohol is selected from a group consisting of ethanol, isopropanol and combinations thereof.

19. A method in accordance with claim 18, wherein the emulsion vehicle contains ethyl laurate, polysorbate 80, propylene glycol, ethanol and water wherein each of ethyl laurate and water comprise 15 percent by weight of the vehicle, and the weight ratio of polysorbate 80, propylene glycol and ethanol is such that the proportion of alcohol is greater than either of the other two.

20. A method in accordance with claim 18, wherein the emulsion vehicle contains about 23.3 weight percent of polysorbate 80 and the weight ratio of polysorbate 80, propylene glycol and ethanol is 1.0:0.86:1.15.

21. A method in accordance with claim 18, wherein the emulsion vehicle contains about 23.3 weight percent of polysorbate 80 and the weight ratio of polysorbate 80, propylene glycol and ethanol is 1.0:0.72:1.29.

22. A method in accordance with claim 18, wherein the emulsion vehicle contains about 20 weight percent of polysorbate 80 and the weight ratio of polysorbate 80, propylene glycol and ethanol is 1.0:1.0:1.5.
